(19) United States
(12) Patent Application Publication
(10) Pub. No.: US 2014/0248629 A1
(43) Pub. Date: Sep. 4, 2014

(54) GOLD NANOPARTICLE BASED DIPSTICK NANO-BIOSENSOR FOR DETECTING PLASMODIUM FALCIPARUM AND PLASMODIUM VIVAX AND MEHTOD OF SYNTHESIZING THE SAME

(71) Applicants: Morteza Mahmoudi, (US); Mohammd Ali Shokrgozar, (US); Pasteur Institute of Iran, Tehran (IR)

(72) Inventors: NAVID DINPARAST DJADID, TEHRAN (IR); FARZANEH SEDIGHIAN, TEHRAN (IR); ABBASALI RAZ, TEHRAN (IR); SEDIGHEH ZAKERI, TEHRAN (IR)

(73) Assignees: Morteza Mahmoudi, Tehran (IR); Pasteur Institute of Iran, Tehran (IR); Mohammd Ali Shokrgozar, Tehran (IR)

(21) Appl. No.: 13/783,330

(22) Filed: Mar. 3, 2013

(51) Int. Cl. C12Q 1/04

(52) U.S. Cl. CPC C12Q 1/04
USPC 435/6.15; 435/91.2

(57) ABSTRACT

The embodiments herein provide a dipstick nano-biosensor for diagnosing Plasmodium vivax and Plasmodium falciparum. The dipstick biosensor comprises a backing plate coated with a cellulose membrane, nitrocellulose membrane and fibreglass. Gold nanoparticles coated with antidigoxigenin are immobilized on the dipstick along with probes comprising strptavidin, texas red, biotin and fluorescein. The dipstick biosensor has three regions comprising a wicking pad, conjugate pad having two control lines and two test lines and an absorbent pad. The first Control line comprises nitrocellulose membrane coated with antifluorescein. The second control line comprises nitrocellulose membrane coated with anti anti-sheep. The first test line comprises nitrocellulose membrane coated with streptavidin conjugated to biotin. The second test line comprises nitrocellulose membrane coated with anti texas red. The two test lines help to confirm the diagnostic results.

### BACKGROUND

1. Technical field

The embodiments herein generally relate to the field of biosensors and particularly to the nano-biosensors used for diagnosing diseases. The embodiments herein more particularly relate to a method of detecting malarial parasites by using a dipstick type nano-biosensor. The embodiments herein further relate to a method of detecting Plasmodium vivax and Plasmodium falciparum.

2. Description of the Related Art

Arthropod-borne infections, such as malaria which are transmitted by mosquitoes, are often quite serious resulting in a significant morbidity and even death. The figures released by the World Health Organization in the World Health Report -1996 shows that 2.1 million deaths were caused by malaria alone in 1995 (Day, “Scourge of infections Kills Third World's Young” in New Scientist, 150(2031): p6, 1996).

Malaria is a mosquito borne infectious disease of humans and other animals and is caused by protists of the genus Plasmodium. A protozoan of the genus Plasmodium, which includes P. falciparum, P. vivax, P. ovate and P. malariae, causes malaria in humans. P. falciparum, which can result in a potentially fatal infection, is widespread throughout the tropics and therefore constitutes an important health threat/hazard for millions of people. P. vivax is also widespread and because of its propensity for successive relapse from liver and consequent toxicity, it constitutes an important cause of morbidity in tropical regions. P. ovale and P. malariae are less common and both of them cause low-grade, chronic diseases and the latter infection often causes a disruption of kidney function through immune-complex deposition.

These protists first infect liver and then act as parasites within Red Blood Cells (RBC) causing symptoms that typically include fever and headache in severe cases progressing to coma or death.

Accurate methods for detecting malaria and other arthropod-borne infections are necessary to identify the infected individuals so as to properly direct a therapy and for identifying further sources that may increase a spread of diseases. The standard and most cost-effective method for detecting the malaria pathogens in mosquitoes involves an isolation of sporozoites from the salivary glands and an enumeration using a phase microscopy on a haemocytometer. However, the method is not very specific and is very much labor intensive. This method requires an effective microscope and skilled technicians. This method is not generally feasible in many regions of the tropics.

The malaria is typically diagnosed by a microscopic examination of blood using blood films. The malaria is also diagnosed by using the antigen based Rapid Diagnostic Tests (RDTs) such as dipsticks and Polymerase Chain Reaction (PCR) based tests.

The microscopic examination of a blood smear reveals the presence of the malarial parasites inside the blood cells. The blood is the most frequently used sample for diagnosis as an invasive specimen while the saliva and the urine have also been investigated as an alternative forming non invasive specimens. But experienced microbiologists and skilled persons are required for these methods. Although the polymerase chain reaction (PCR) based tests have been developed, these are not widely implemented in malarial endemic regions due to their complexity.

The dipsticks are a new generation of antigen based rapid diagnostics tests which are sensitive and the dip sticks are a reliable device for the diagnosis of the malaria. Initially the glutamate dehydrogenase of the P. falciparum was used as an antigen. It is now replaced by the lactate dehydrogenase. The nucleic acid sequence based application (NASBA) products (RNA nucleotides) are used along with antibodies that are labeled with the digoxigenin-uridine triphosphate (dig-UTP). The labeled NASBA products are coated on the nanoparticles. The dipstick based tests allow a rapid diagnosis of the malaria by people who are not otherwise skilled in the laboratory techniques for diagnosing the malarial parasites. Also the dipsticks find their application in field diagnosis where there are no diagnosis centers. None of the rapid tests are currently as sensitive as thick blood film, or cheap. A major drawback in the use of all current dipstick methods is that the result is qualitative.

The U.S. Pat. No. 7,427,483 discloses PCR reaction for amplification and detection of Plasmodium falciparum DNA (a highly conserved region of Plasmodium falciparum MSP1 gene) and final detection is done by Enzyme Linked Immunosorbent Assay (ELISA) procedure. Further the patent discloses the use of polymerase chain reaction (PCR) to label products. The method is specific for C-terminal region of MSP 1 gene. Because of the method and protocol of ELISA, this method requires more time for delivering the results and hence a diagnosis will be delayed. Such a method cannot cope with a great number of infected persons in the area where malaria is prevalent and the number of inspection experts is insufficient.

Another prior art by the U.S. patent No. 2011/0171749A1 discloses the biosensor based on antigen-antibody reaction. Further the biosensor is mainly designed for detecting the Escherichia coli and utilizes polymer coated magnetic nanoparticles. This method is specific to bacterial detection and not for malarial parasite detection. Further this method requires magnetic field which makes the diagnosis complex.

Yet another prior art by U.S. Pat. No. 5,827,681 discloses light microscope for detecting opaque hemozoin particles and has used multiple champers. Hence this method requires microbiological skills. Further the method discloses a use of microscope which limits the use of the invention in laboratory and not in the field conditions. Such a method cannot cope with a great number of infected persons in the area where malaria is prevalent and the number of inspection experts is insufficient.

Yet another prior art by U.S. Pat. No. 5,792,609 discloses a use of a primer for detecting the malarial parasites which is not labeled and hence the method makes it difficult to detect the binding of template sequence and the primer. Hence a monitoring of the primer and template sequence binding to provide a desired result is not assured.

According to yet another prior art, the immune sensor is based on the antigen-antibody reaction. The immune sensor detects the HRP-2 of the Plasmodium falciparum in blood. It uses the disposable screen printed electrodes (SPE's) modified with multiwall carbon nanotubes and the gold nanoparticles. This makes the invention complex as the electrodes are used. Further the article discloses that the final results are obtained based on the microscopic results. Hence the biosensor cannot be used in the field conditions or the places where epidemic of malaria is prevailing.

Hence, there is a need to develop a device which is simple, efficient and sensitive, and which does not require a use of any other instrument while handling. Also, there is need to develop a device that does not require microbiological skills especially in places where there are no diagnostic centers. Further simple assays are much needed in the art to improve the availability of malarial diagnosis. Also there is a need to provide better treatment options to those in need, and to differentiate between symptomatic and asymptomatic disease.

The above mentioned shortcomings, disadvantages and problems are addressed herein and which will be understood by reading and studying the following specification.

### OBJECTIVES OF THE EMBODIMENTS

The primary object of the embodiments herein is to provide a dipstick nano-biosensor with the gold nanoparticles conjugated to an antibody for diagnosis of the malarial parasites.

Another object of the embodiments herein is to provide a dipstick nano-biosensor for diagnosis of the Plasmodium falciparum and Plasmodium vivax.

Yet another object of the embodiment herein is to provide dipstick nano-biosensor with 90% sensitivity for detecting the Plasmodium falciparum and Plasmodium vivax.

Yet another object of the embodiments herein is to provide sensitive and reliable method for diagnosing malaria in the field conditions, where the diagnostic centers are not present.

Yet another object of the embodiment herein is to provide a potable and a sensitive dipstick nano-biosensor device.

Yet another object of the embodiment herein is to combine a molecular method of NASBA (Nucleic Acid Sequence Based Amplification) with the nanotechnology (use of gold nanoparticles) for preparing the dipstick nano-biosensor.

Yet another object of the embodiments herein is to provide a dipstick nano-biosensor based on the amplification of 18 srRNA by NASBA method.

Yet another object of the embodiments herein is to label NASBA products with the Digoxigenin-11-UTP.

Yet another object of the embodiments is to conjugate the labeled NASBA products with anti digoxigenin conjugated-nano gold particles.

Yet another object of the embodiments herein is to provide a dipstick nano-biosensor comprising two control zones for confirming the diagnostic results of the malarial parasites.

Yet another object of the embodiments herein is to provide a dipstick nano-biosensor so that the results are observed after performing oligochromatography and a microscope is not needed.

These and other objects and advantages of the embodiments herein will become readily apparent from the following detailed description taken in conjunction with the accompanying drawings.

### SUMMARY

The various embodiments herein provide a gold nanoparticle based dipstick nano-biosensor for detecting plasmodium falciparum and plasmodium vivax and a mehtod of synthesizing the same. The embodiments herein provide a new innovative method for a diagnosis of the malarial parasites. The gold nanoparticles based dipstick nano-biosensor is used in a new generation of antigen based rapid and sensitive diagnostic test, and the dipstick nano bio sensor is a reliable device for diagnosing the Plasmodium vivax and Plasmodium falciparum.

According to an embodiment herein, a dipstick nano-biosensor is provided for detecting Plasmodium vivax and Plasmodium falciparum. The dipstick nano-bio sensor comprising a cellulose membrane, a nitrocellulose membrane, a fibreglass, and a plurality of probes. The cellulose membrane, the nitrocellulose membrane and the fiber glass are coated on a backing plastic plate coated with gold nanoparticles conjugated with an anti-body and wherein the anti-body is an anti-digoxigenin.

According to an embodiment herein, the cellulose and the nitrocellulose membrane are coated on the backing plastic plate with an overlap of 1 mm.

According to an embodiment herein, the gold nanoparticles are conjugated to the anti digoxigenin antibody by a physical adsorption for 1.5 hours in a cold room by a continuous mixing of the gold nanoparticles, and wherein a size of the gold nanoparticles is 0.40-25nm.

According to an embodiment herein, the plurality of the probes comprises a streptavidin, a texas red, a biotin and a fluoresceine.

According to an embodiment herein, the backing plastic plate comprises three regions and wherein the three regions comprise a wicking pad, a conjugate pad, and an absorbent pad respectively.

According to an embodiment herein, the conjugate pad comprises a test zone and a control zone, and wherein the test zone comprises a first test line and a second test line for detecting Plasmodium falciparum and Plasmodium vivax respectively, and wherein the control zone comprises a first control zone and a second control line for checking a correctness of NASBA and oligo chromatography respectively.

According to an embodiment herein, the first test line comprises a nitrocellulose membrane coated with a streptavidin conjugated to a biotin and wherein the second test line comprises a nitrocellulose membrane coated with an anti texas red.

According to an embodiment herein, the first control line comprises a nitrocellulose membrane coated with an antifluorescene antibody and wherein the second control line comprises a nitrocellulose membrane coated with an anti anti-sheep.

According to an embodiment herein, the plurality of probes comprises a 18srRNA and wherein the 18srRNA is isolated from Plasmodium falciparum, Plasmodium vivax and a GAPDH, and wherein the GAPDH is isolated from a blood of healthy human individual, and wherein the isolated 18srRNA and the GAPDH are subjected to an agarose gel electrophoresis to extract specific bands with sequences from an agarose gel.

According to an embodiment herein, the 18srRNA is subjected to a Reverse Transcriptase Polymerase Chain Reaction (RT-PCR) to get a cDNA.

According to an embodiment herein, the isolated sequences from the agarose gel are cloned into a plasmid vector, and wherein the cloned plasmid vectors are induced into an Escherichia coli, and wherein the plasmids are sequenced.

According to an embodiment herein, 18srRNA and GAPDH are subjected to Nucleic Acid Sequence Based Amplification (NASBA), and wherein NASBA amplified 18sRNA and GAPDH are labelled with digoxigenin-11-UTP.

According to an embodiment herein, the backing plastic plate is coated with digoxigenin-11-UTP labelled 18srRNA, streptavidin, anti texas red, biotin and fluorescein by Airjet 3000.

According to an embodiment herein, a method is provided for synthesizing dipstick nano-biosensor for detecting Plasmodium vivax and Plasmodium falciparum. The method comprises culturing Plasmodium falciarum and Plasmodium vivax, extracting RNA from Plasmodium falciparum and Plasmodium vivax culture and a healthy donor, cloning and sequencing extracted RNA using a Reverse Transcriptase Polymerase Chain Reaction (RT-PCR) to get a cDNA, performing In vitro transcription of extracted RNA, preparing control positive RNA, isolating GAPDH from a blood of healthy human individual, setting and performing NASBA-DIG labelling of both Pasmodium falciparum and Plasmodium vivax and GAPDH, selecting a plurality of membranes for assembling a dipstick, and wherein the plurality of membranes includes a cellulose membrane, a nitrocellulose membrane, and a fiberglass, and conjugating nano gold particles to anti digoxigenin antibody to form a dipstick.

According to one embodiment herein, the first step for the preparation of a dipstick nano-biosensor and the detection of malarial parasites is a preparation of positive control by an amplification of the RNA's by Nucleic Acid Sequence Based Amplification (NASBA). This step further comprises of RNA extraction, subjecting the isolated RNA's to RT-PCR, cloning the RT-PCR products, sequencing the genes and in vitro transcription. The two species of malarial parasites a Plasmodium falciparum and a Plasmodium vivax are cultured in Pasteur Institute of Iran. The 18 srRNA is isolated from Plasmodium vivax, Plasmodium falciparum continuous invitro culture and healthy human blood. Before subjecting the isolated RNA's to Reverse Transcription—Polymerase Chain Reaction (RT-PCR), the specific probes and the primers are designed in such a way that each specific primer pair does not detect the other species. Further a pair of primers for Glyceraldehyde 3-phosphate dehydrogenase (GAPDH) gene, which is a house keeping gene, is designed for checking the correctness of NASBA. The GAPDH gene is co-amplified with the other genes and a specific probe for each gene is designed to detect the NASBA product of each gene.

According to one embodiment herein, the RT-PCR is performed with the specific primers for each gene after designing the specific primers and the probes for each gene. The amplified fragments of a cDNA, thus obtained from the RT-PCR, are cloned into a plasmid vector pDrive (Qiagen) having an ampicillin resistance gene. The cloned plasmid vector pDrive is transformed in the Escherichia coli (DH5). The Escherichia coli are cultivated in LB (Luria Bertani) agar media having an ampicillin, a Xgal (5-bromo-4-chloro-indolyl-β-D-galactopyranoside) and an IPTG (isopropylthio-β-galactoside). The colonies are screened and white colonies are taken for plasmid extraction. The plasmids are sequenced and an existence of desired inserts in them is verified.

According to one embodiment herein, an in vitro transcription is further performed to synthesize the RNAs with same size. Hence a linear DNA molecule is needed as a pattern of the template sequence. In this process, PCR reaction is performed by the specific primers having a T7 promoter sequence. The PCR products, which are linear DNA molecules having the T7 promoter, are used in an in vitro transcription reaction as patterns or templates. After this step Plasmodium falciparum and GAPDH RNA are synthesized in laboratory by using the T7 RNA polymerase enzyme which generates RNA from patterns having the T7 promoter sequence. The RNAs generated by the invitro transcription reaction are treated by a DNase I for removing the DNA molecules and are then purified by the Qiazol lysis buffer and stored in −70 ° C. for further use in the NASBA as pattern or template.

According to one embodiment herein, the next step for the synthesis of dipstick nano-biosensor is the dig-labeling (Digoxigenin- labeling) of NASBA products. Hence the NASBA reaction is set and Dig-11-UTP is used instead of usual UTP. The reason for labeling the NASBA products (RNA) with the digoxigenin molecules is that the RNA is required for detection by the antidigoxigenin molecules conjugated with nano gold particles on dipstick nano-biosensor. The RNA labeled with the digoxigenin-11-UTP is now known as dig-UTP. The dig-UTP is now used with a plurality of probes for the dipstick nano-biosensor. The plurality of probes comprises a biotin, a streptavidin, an antifluoresin, an anti-texas red and an IgG antibody of Sheep.

According to one embodiment herein, the next step is the preparation of reagents for the dipstick nano-biosensor. This step comprises of generating an anti sheep or an anti digoxigenin antibody of sheep, which is a conjugation of an anti digoxigenin with the 40 nm nano gold particles, verification of existing sheep antibodies by sodium dodecyl sulfate polyacrylamide gel electrophoresis (SDS-PAGE) and Western blotting. The probes such as a Texas red, a biotin, a streptavidin are procured.

According to one embodiment herein, the next step is preparation and assembly of a dipstick nano-biosensor. The dipstick nano-biosensor used for detecting malarial parasites of the genus Plasmodium comprises a cellulose, a nitrocellulose and a fiberglass, the gold nanoparticles coated with an anti-digoxigenin, the NASBA products labeled with the dig-11-UTP and the probes. The probes comprise of a streptavidin, a texas red, a biotin and a fluorescein. The cellulose, nitrocellulose and fiberglass are coated on the backing plastic such that they have 1 mm overlap. Further the nano-biosensor dipstick comprises a control region and a test region. The size of the gold nanoparticles is 25 nm. The gold nanoparticles are coated with an anti-digoxigenin to form a ligand on the membrane.

According to one embodiment herein, a suitable membrane is selected for the dipstick nano-biosensor comprising the gold nanoparticles coated with an anti-digoxigenin antibody. The membranes comprise a cellulose, a nitrocellulose and a fiberglass. The membranes are arranged in a manner such that each membrane overlaps the other by 1 mm. The combined mixture of the Dig-UTP labeled RNA and the probes are added to the membrane.

According to one embodiment herein, the dipstick nano-biosensor further comprises a capture reagent which is prepared in optimum or appropriate concentrations. The capture reagents include a streptavidine, an anti fluoresceine which are obtained from Roche and Biolegend companies respectively and the other reagents such as an anti sheep are prepared in laboratory. For preparing the anti sheep the IgG from the sheep blood are purified by PrG column. The verification process for a presence of existing IgG SDS PAGE is performed. Further the rabbits are immunized by the sheep IgG three times (10 μg/ml). The blood from the immunized rabbits is taken and IgG from the rabbit blood is purified by PrG column. The functionality of the anti sheep obtained from the rabbit blood is verified by performing the SDS-PAGE and the western blotting.

According to one embodiment herein, the dipstick is a portable and light device which contain three parts namely a wicking pad, a conjugate pad, and an absorbent pad. These parts are adhered on a backing plastic in a way that they have 1 mm overlap with each other. The nano gold particles of 0.40 nm are conjugated to anti digoxigenin and are loaded on conjugate pad of fiber glass. The streptavidine , anti fluorsceine and anti anti sheep are loaded on the membrane by air jet 3000 in order to form a test line, a first control line and a second control line respectively.

According to one embodiment herein, the utility and application of the dipstick nano-biosensor is done by the visualization of bands by an oligochromatography. For the oligochromatography process, a proper concentration of each probe and various dilutions of the probes are prepared.

The digoxigenin labeled NASBA products for each gene is mixed with their specific probes and after the hybridization is complete for 10 minutes at 25° C. After hybridization, the mixture is loaded on the conjugate pad. The wicking pad is dipped into hybridization buffer after loading the conjugate pad. The red lines at the bottom of dipstick nano-biosensor shows that the dipstick nano-biosensor can detect hybridization product of Plasmodium falciparum 18srRNA (NASBA product+specific probes). The red line at the top of the dipstick shows that the gold nanoparticles conjugated to the anti digoxigenin are moved along the dipstick nao-biosensor and the gold nanoparticles conjugated to anti digoxigenin could react with the anti sheep coated on the second control line.

The dipstick has detected hybridization product of the Plasmodium falciparum 18srRNA (NASBA product+specific probes) and also hybridization product of the GAPDH (NASBA product+specific probes).

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a flow chart indicating a method of preparation and a diagnostic test performance of a dipstick nano-biosensor, according to one embodiment herein.

FIG. 2 illustrates a flow chart indicating a method of preparation of a positive control for NASBA, according to one embodiment herein.

FIG. 3 illustrates a flow chart indicating a method of isolation of the RNA from blood, according to one embodiment herein.

FIG. 4 illustrates a flow chart indicating a method of cloning and screening of transformed Escherichia coli, according to one embodiment herein.

FIG. 5 shows an image exhibiting the white blue colony screening of 18srRNA gene after transformation, according to one embodiment herein.

FIG. 6 illustrates a flow chart indicating an invitro transcription for a generation of RNA from cDNA, according to one embodiment herein.

FIG. 7 illustrates a flow chart indicating a Digoxigenin-11-UTP labeling of NASBA products i.e. RNA, according to one embodiment herein.

FIG. 8 shows an image exhibiting the agarose gel electrophoresis of Plasmodium falciparum 18srRNA and the NASBA dig-labeling of P. falciparum 18srRNA, according to one embodiment herein.

FIG. 9 shows an image exhibiting an agarose gel electrophoresis of NASBA for the Plasmodium falciparum 18srRNA and a GAPDH, according to one embodiment herein.

FIG. 10 shows an image exhibiting an agarose gel electrophoresis of invitro transcription of a GAPDH gene, according to one embodiment herein.

FIG. 11 shows an image exhibiting an agarose gel electrophoresis of the 18srRNA of Plasmodium vivax, Plasmodium falciparum and GAPDH isolated from healthy human blood obtained after RT-PCR, according to one embodiment herein.

FIG. 12 shows an image exhibiting an agarose gel electrophoresis of GAPDH, Plasmodium falciparum 18srRNA and Plasmodium vivax 18srRNA obtained after NASBA, according to one embodiment herein.

FIG. 13 illustrates a flow chart indicating the anti sheep preparation for loading on dipstick nano-biosensor, according to one embodiment herein.

FIG. 14 shows an image exhibiting the SDS-PAGE of sheep

IgG's after purification by PrG column before immunizing a rabbit, according to one embodiment herein.

FIG. 15 is schematic view of the dipstick nano-biosensor assembly, according to one embodiment herein.

FIG. 16a illustrates a screen shot indicating the designing of the specific primers for Plasmodium vivax, according to one embodiment herein.

FIG. 16b illustrates a screen shot indicating the designing of the specific primers for the Plasmodium falciparum, according to one embodiment herein.

FIG. 16c illustrates a screen shot indicating the designing of the specific primers for the human GAPDH, according to one embodiment herein.

FIG. 17 illustrates a flow chart indicating an oligochromatography process, according to one embodiment herein.

FIG. 18 shows an image exhibiting a first test line and a second control line in dipstick nano-biosensor, according to one embodiment herein.

FIG. 19 shows an image exhibiting a hybridization product of the Plasmodium falciparum 18srRNA and the human GAPDH, according to one embodiment herein.

FIG. 20 shows a result of a Plasmodium falciparum 18srRNA gene sequencing process, according to one embodiment herein.

FIG. 21 shows a result of a human GAPDH gene sequencing process, according to one embodiment herein.

FIG. 22 shows a screen shot illustrating a Blast analysis of amplified sequence of Plasmodium falciparum 18srRNA, according to one embodiment herein.

FIG. 23 shows a screen shot illustrating a Blast analysis of amplified sequence of Plasmodium vivax 18srRNA, according to one embodiment herein.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a gold nanoparticle based dipstick nano-biosensor for detecting plasmodium falciparum and plasmodium vivax and a mehtod of synthesizing the same. The embodiments herein provide a new innovative method for a diagnosis of the malarial parasites. The gold nanoparticles based dipstick nano-biosensor is used in a new generation of antigen based rapid and sensitive diagnostic test, and the dipstick nano bio sensor is a reliable device for diagnosing the Plasmodium vivax and Plasmodium falciparum.

According to an embodiment herein, a dipstick nano-biosensor is provided for detecting Plasmodium vivax and Plasmodium falciparum. The dipstick nano-bio sensor comprising a cellulose membrane, a nitrocellulose membrane, a fibreglass, and a plurality of probes. The cellulose membrane, the nitrocellulose membrane and the fiber glass are coated on a backing plastic plate coated with gold nanoparticles conjugated with an anti-body and wherein the anti-body is an anti-digoxigenin.

According to an embodiment herein, the cellulose and the nitrocellulose membrane are coated on the backing plastic plate with an overlap of 1 mm.

According to an embodiment herein, the gold nanoparticles are conjugated to the anti digoxigenin antibody by a physical adsorption for 1.5 hours in a cold room by a continuous mixing of the gold nanoparticles, and wherein a size of the gold nanoparticles is 0.40-25 nm.

According to an embodiment herein, the plurality of the probes comprises a streptavidin, a texas red, a biotin and a fluoresceine.

According to an embodiment herein, the backing plastic plate comprises three regions and wherein the three regions comprise a wicking pad, a conjugate pad, and an absorbent pad respectively.

According to an embodiment herein, the conjugate pad comprises a test zone and a control zone, and wherein the test zone comprises a first test line and a second test line for detecting Plasmodium falciparum and Plasmodium vivax respectively, and wherein the control zone comprises a first control zone and a second control line for checking a correctness of NASBA and oligo chromatography respectively.

According to an embodiment herein, the first test line comprises a nitrocellulose membrane coated with a streptavidin conjugated to a biotin and wherein the second test line comprises a nitrocellulose membrane coated with an anti texas red.

According to an embodiment herein, the first control line comprises a nitrocellulose membrane coated with an antifluorescene antibody and wherein the second control line comprises a nitrocellulose membrane coated with an anti anti-sheep.

According to an embodiment herein, the plurality of probes comprises a 18srRNA and wherein the 18srRNA is isolated from Plasmodium falciparum, Plasmodium vivax and a GAPDH, and wherein the GAPDH is isolated from a blood of healthy human individual, and wherein the isolated 18srRNA and the GAPDH are subjected to an agarose gel electrophoresis to extract specific bands with sequences from an agarose gel.

According to an embodiment herein, the 18srRNA is subjected to a Reverse Transcriptase Polymerase Chain Reaction (RT-PCR) to get a cDNA.

According to an embodiment herein, the isolated sequences from the agarose gel are cloned into a plasmid vector, and wherein the cloned plasmid vectors are induced into an Escherichia coli, and wherein the plasmids are sequenced.

According to an embodiment herein, 18srRNA and GAPDH are subjected to Nucleic Acid Sequence Based Amplification (NASBA), and wherein NASBA amplified 18sRNA and GAPDH are labelled with digoxigenin-11-UTP.

According to an embodiment herein, the backing plastic plate is coated with digoxigenin-11-UTP labelled 18srRNA, streptavidin, anti texas red, biotin and fluorescein by Airjet 3000.

According to an embodiment herein, a method is provided for synthesizing dipstick nano-biosensor for detecting Plasmodium vivax and Plasmodium falciparum. The method comprises culturing Plasmodium falciarum and Plasmodium vivax, extracting RNA from Plasmodium falciparum and Plasmodium vivax culture and a healthy donor, cloning and sequencing extracted RNA using a Reverse Transcriptase Polymerase Chain Reaction (RT-PCR) to get a cDNA, performing In vitro transcription of extracted RNA, preparing control positive RNA, isolating GAPDH from a blood of healthy human individual, setting and performing NASBA-DIG labelling of both Pasmodium falciparum and Plasmodium vivax and GAPDH, selecting a plurality of membranes for assembling a dipstick, and wherein the plurality of membranes includes a cellulose membrane, a nitrocellulose membrane, and a fiberglass, and conjugating nano gold particles to anti digoxigenin antibody to form a dipstick.

According to one embodiment herein, the first step for the preparation of a dipstick nano-biosensor and the detection of malarial parasites is a preparation of positive control by an amplification of the RNA's by Nucleic Acid Sequence Based Amplification (NASBA). This step further comprises of RNA extraction, subjecting the isolated RNA's to RT-PCR, cloning the RT-PCR products, sequencing the genes and in vitro transcription. The two species of malarial parasites a Plasmodium falciparum and a Plasmodium vivax are cultured in Pasteur Institute of Iran. The 18 srRNA is isolated from Plasmodium vivax, Plasmodium falciparum continuous invitro culture and healthy human blood. Before subjecting the isolated RNA's to Reverse Transcription—Polymerase Chain Reaction (RT-PCR), the specific probes and the primers are designed in such a way that each specific primer pair does not detect the other species. Further a pair of primers for Glyceraldehyde 3-phosphate dehydrogenase (GAPDH) gene, which is a house keeping gene, is designed for checking the correctness of NASBA. The GAPDH gene is co-amplified with the other genes and a specific probe for each gene is designed to detect the NASBA product of each gene.

According to one embodiment herein, the RT-PCR is performed with the specific primers for each gene after designing the specific primers and the probes for each gene. The amplified fragments of a cDNA, thus obtained from the RT-PCR, are cloned into a plasmid vector pDrive (Qiagen) having an ampicillin resistance gene. The cloned plasmid vector pDrive is transformed in the Escherichia coli (DH5). The Escherichia coli are cultivated in LB (Luria Bertani) agar media having an ampicillin, a Xgal (5-bromo-4-chloro-indolyl-f3-D-galactopyranoside) and an IPTG (isopropylthio-f3-galactoside). The colonies are screened and white colonies are taken for plasmid extraction. The plasmids are sequenced and an existence of desired inserts in them is verified.

According to one embodiment herein, an in vitro transcription is further performed to synthesize the RNAs with same size. Hence a linear DNA molecule is needed as a pattern of the template sequence. In this process, PCR reaction is performed by the specific primers having a T7 promoter sequence. The PCR products, which are linear DNA molecules having the T7 promoter, are used in an in vitro transcription reaction as patterns or templates. After this step Plasmodium falciparum and GAPDH RNA are synthesized in laboratory by using the T7 RNA polymerase enzyme which generates RNA from patterns having the T7 promoter sequence. The RNAs generated by the invitro transcription reaction are treated by a DNase I for removing the DNA molecules and are then purified by the Qiazol lysis buffer and stored in −70 ° C. for further use in the NASBA as pattern or template.

According to one embodiment herein, the next step for the synthesis of dipstick nano-biosensor is the dig-labeling (Digoxigenin-labeling) of NASBA products. Hence the NASBA reaction is set and Dig-11-UTP is used instead of usual UTP. The reason for labeling the NASBA products (RNA) with the digoxigenin molecules is that the RNA is required for detection by the antidigoxigenin molecules conjugated with nano gold particles on dipstick nano-biosensor. The RNA labeled with the digoxigenin-11-UTP is now known as dig-UTP. The dig-UTP is now used with a plurality of probes for the dipstick nano-biosensor. The plurality of probes comprises a biotin, a streptavidin, an antifluoresin, an anti-texas red and an IgG antibody of Sheep.

According to one embodiment herein, the next step is the preparation of reagents for the dipstick nano-biosensor. This step comprises of generating an anti sheep or an anti digoxigenin antibody of sheep, which is a conjugation of an anti digoxigenin with the 40 nm nano gold particles, verification of existing sheep antibodies by sodium dodecyl sulfate polyacrylamide gel electrophoresis (SDS-PAGE) and Western blotting. The probes such as a Texas red, a biotin, a streptavidin are procured.

According to one embodiment herein, the next step is preparation and assembly of a dipstick nano-biosensor. The dipstick nano-biosensor used for detecting malarial parasites of the genus Plasmodium comprises a cellulose, a nitrocellulose and a fiberglass, the gold nanoparticles coated with an anti-digoxigenin, the NASBA products labeled with the dig-11-UTP and the probes. The probes comprise of a streptavidin, a texas red, a biotin and a fluorescein. The cellulose, nitrocellulose and fiberglass are coated on the backing plastic such that they have 1 mm overlap. Further the nano-biosensor dipstick comprises a control region and a test region. The size of the gold nanoparticles is 25 nm. The gold nanoparticles are coated with an anti-digoxigenin to form a ligand on the membrane.

According to one embodiment herein, a suitable membrane is selected for the dipstick nano-biosensor comprising the gold nanoparticles coated with an anti-digoxigenin antibody. The membranes comprise a cellulose, a nitrocellulose and a fiberglass. The membranes are arranged in a manner such that each membrane overlaps the other by 1 mm. The combined mixture of the Dig-UTP labeled RNA and the probes are added to the membrane.

According to one embodiment herein, the dipstick nano-biosensor further comprises a capture reagent which is prepared in optimum or appropriate concentrations. The capture reagents include a streptavidine, an anti fluoresceine which are obtained from Roche and Biolegend companies respectively and the other reagents such as an anti sheep are prepared in laboratory. For preparing the anti sheep the IgG from the sheep blood are purified by PrG column. The verification process for a presence of existing IgG SDS PAGE is performed. Further the rabbits are immunized by the sheep IgG three times (10 μg/ml). The blood from the immunized rabbits is taken and IgG from the rabbit blood is purified by PrG column. The functionality of the anti sheep obtained from the rabbit blood is verified by performing the SDS-PAGE and the western blotting.

According to one embodiment herein, the dipstick is a portable and light device which contain three parts namely a wicking pad, a conjugate pad, and an absorbent pad. These parts are adhered on a backing plastic in a way that they have 1 mm overlap with each other. The nano gold particles of 0.40 nm are conjugated to anti digoxigenin and are loaded on conjugate pad of fiber glass. The streptavidine , anti fluorsceine and anti anti sheep are loaded on the membrane by air jet 3000 in order to form a test line, a first control line and a second control line respectively.

According to one embodiment herein, the utility and application of the dipstick nano-biosensor is done by the visualization of bands by an oligochromatography. For the oligochromatography process, a proper concentration of each probe and various dilutions of the probes are prepared. The digoxigenin labeled NASBA products for each gene is mixed with their specific probes and after the hybridization is complete for 10 minutes at 25° C. After hybridization, the mixture is loaded on the conjugate pad. The wicking pad is dipped into hybridization buffer after loading the conjugate pad. The red lines at the bottom of dipstick nano-biosensor shows that the dipstick nano-biosensor can detect hybridization product of Plasmodium falciparum 18srRNA (NASBA product+specific probes). The red line at the top of the dipstick shows that the gold nanoparticles conjugated to the anti digoxigenin are moved along the dipstick nao-biosensor and the gold nanoparticles conjugated to anti digoxigenin could react with the anti sheep coated on the second control line. The dipstick has detected hybridization product of the Plasmodium falciparum 18srRNA (NASBA product+specific probes) and also hybridization product of the GAPDH (NASBA product+specific probes).

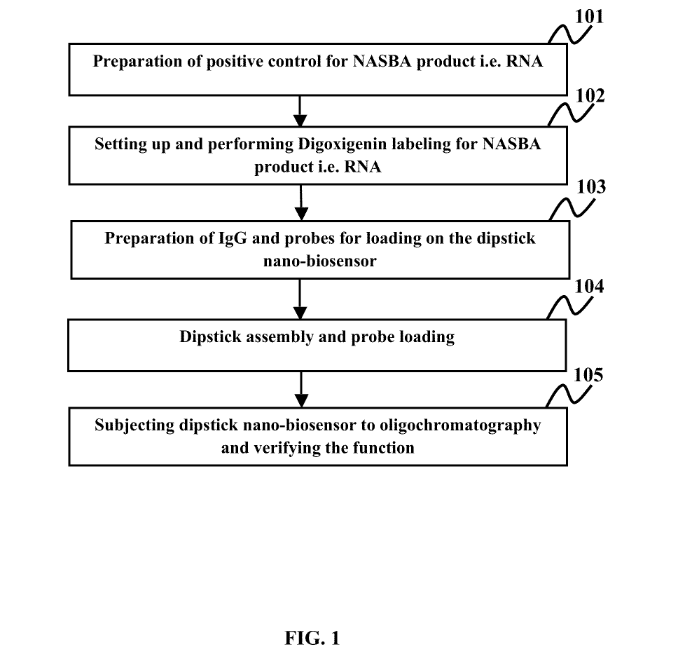

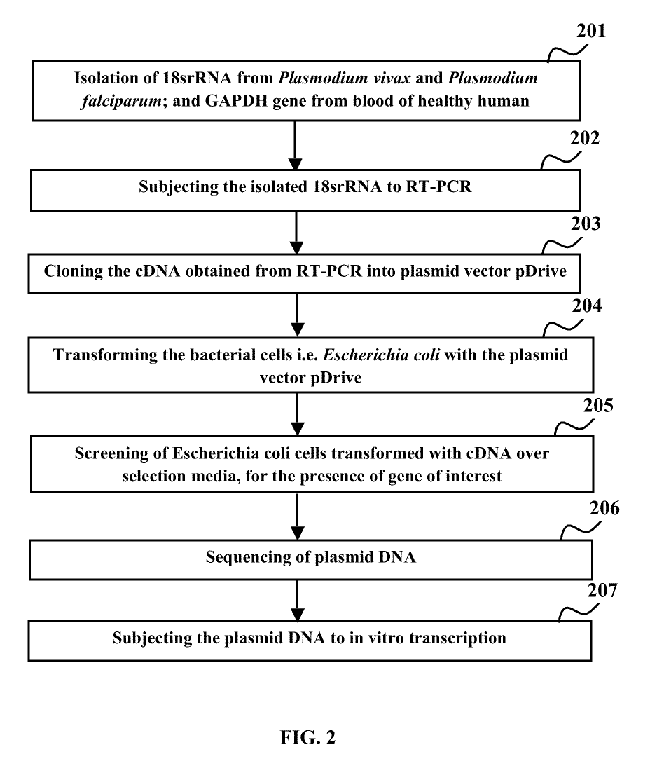

FIG. 1 illustrates a flow chart indicating the preparation and diagnostic test performance of a dipstick nano-biosensor, according to one embodiment herein. The preparation of the dipstick nano-biosensor and the diagnosis of the malarial parasites start with a preparation of a positive control for Nucleic Acid Sequence Based Amplification (NASBA) (101). The isolated RNA is amplified with NASBA in the first step. The second step in the dipstick nano-biosensor preparation is setting up and performing the digoxigenin labeling of NASBA amplified RNA (102). The NASBA products (RNA's) are Digoxigenin labeled as the gold nanoparticles associated anti-digoxigein antibody binds with the Digoxigenin and makes it easier for diagnosis. The third step in the preparation of dipstick nano-biosensor is the preparation of probes like a biotin, a streptavidin and a Texas red; and the assembly of different layers of dipstick nano-biosensor (103). The probes prepared are loaded on the different zones and layers of the dipstick nano-biosensor. The dipstick nano-biosonsor is loaded with all the probes, gold nanoparticles associated anti-digoxigenin and the NASBA digoxigenin labeled RNA's (104). The last step is subjecting the dipstick nano-biosensor for oligochromatography for diagnosis of malarial parasites (105).

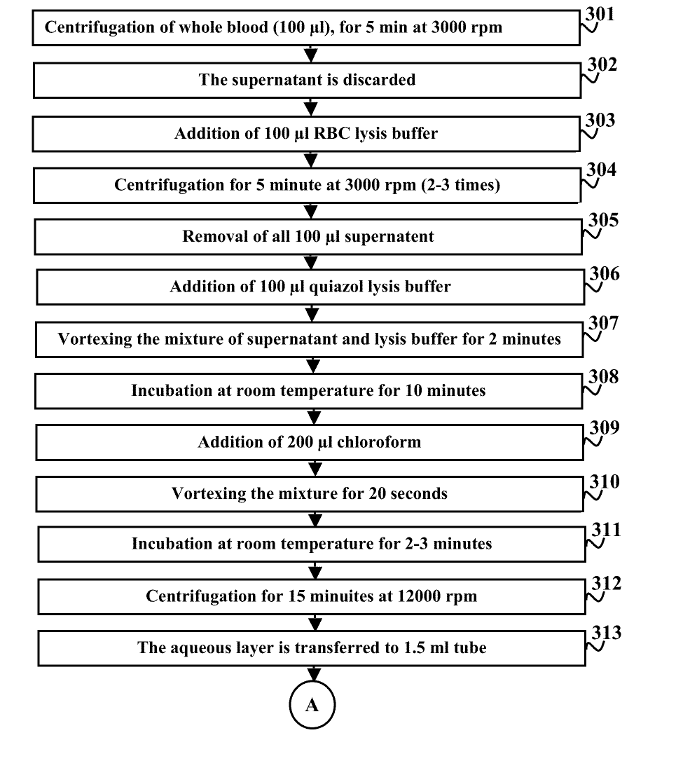

FIG. 2 illustrates a flow chart indicating a method of preparation of positive control for nucleic acid sequence based amplification (NASBA), according to one embodiment herein. The preparation of positive control for the NASBA starts with the isolation of 18srRNA from the invitro continuous culture of the Plasmodium vivax and Plasmodium falciparum and the isolation of a GAPDH from blood of healthy human (201). The isolated 18srRNA is then subjected to reverse transcription polymerase chain reaction (RT-PCR) (202). The cDNA is obtained From RT-PCR and cDNA is subjected to cloning in a plasmid vector pDrive (203). The cloning is done to confirm the presence 18srRNA. The Escherichia coli bacterium is transformed with the pDrive plasmid containing the cDNA of 18srRNA for confirming the presence of the 18srRNA in isolated RNA, (204). The Escherichia coli bacterium after transformation is inoculated on the selection media containing the X-Gal (5-bromo-4-chloro-indolyl-f3-D-galactopyranoside), the IPTG (Isopropyl f3-D-1-thiogalactopyranoside) and the ampicillin (205). The transformed colonies with cDNA plasmid appear to be white in color, and are taken for sequencing of plasmid DNA (206). After the plasmids are sequenced, the plasmids are subjected to the in vitro transcription for producing the RNA (207).

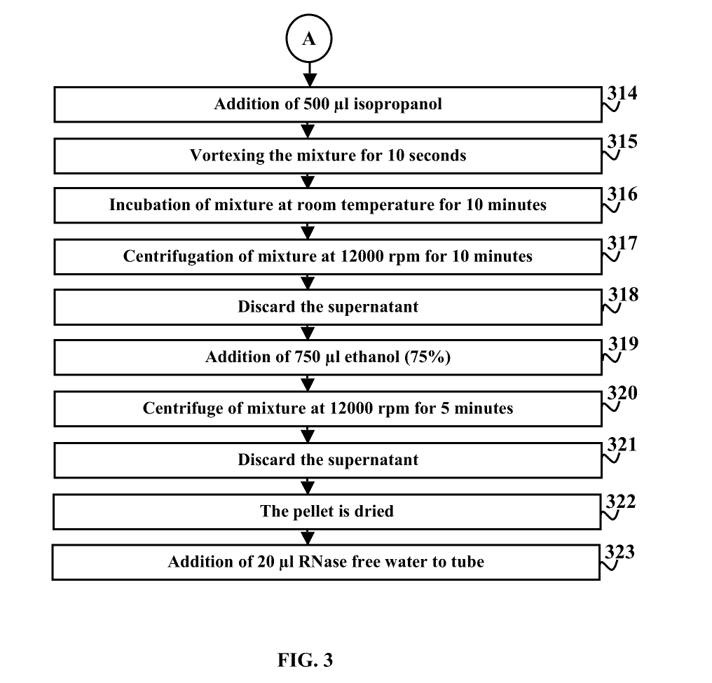

FIG. 3 illustrates a flow chart explaining the method of isolation of RNA from the blood, according to one embodiment herein. The RNA extraction is carried out continuously from the invitro cultures of Plasmodium vivax and Plasmodium falciparum maintained at Pasteur Institute of Iran and GAPDH is isolated from the healthy human blood by qiazol lysis buffer. The GAPDH gene is a house keeping gene which is used for checking the correctness of NASBA. This is the first step for NASBA. The 100 μl of blood is subjected to a centrifugation for 5 minutes at 3000 rpm (301). The supernatant is discarded (302). After discarding the supernatant, 100 μl of RBC lysis buffer is added (303). After the addition of the RBC lysis buffer, the solution is subjected to a centrifugation for 5 minutes at 3000 rpm, and this process is repeated for 3-4 times (304). About 100 μl of the supernatant is again removed (305). In the next step, a qiazol lysis buffer is added to get a mixture (306). The mixture is further subjected to vortexing for 2 minutes (307). Further the mixture is incubated at a room temperature for 10 minutes (308). After incubation, 200 μl of chloroform is added (309). After the addition of chloroform, the mixture is subjected to vortexing for 20 seconds (310). The next step is an incubation of the mixture at room temperature for 2-3 minutes (311). The mixture is then subjected to a centrifugation for 15 minutes at 12000 rpm (312). The aqueous layer (solution) obtained after the centrifugation is transferred to a new tube of 1.5 ml capacity (313). About 500 μl of isopropanol is added to the aqueous solution to get a mixture (314). The mixture obtained after addition of isopropanol is subjected to vortexing for 10 seconds (315). The mixture is incubated at room temperature for 10 minutes (316). The mixture is subjected to centrifugation for 10 minutes at 12000 rpm (317). After centrifugation, the supernatant is discarded (318). After the supernatant is discarded, 750 μl of 75% ethanol is added to the residue in the tube (319). The tube is then subjected to centrifugation for 5 minutes at 12000 rpm (320). After centrifugation, the supernatant is discarded (321). The pellet in the tube is dried (322). After drying the pellet, 20 μl of RNase free water is added to suspend the pellet containing the RNA (323). Hence 160 by and 180 by 18srRNA are isolated from the Plasmodium vivax and the Plasmodium falciparum respectively.

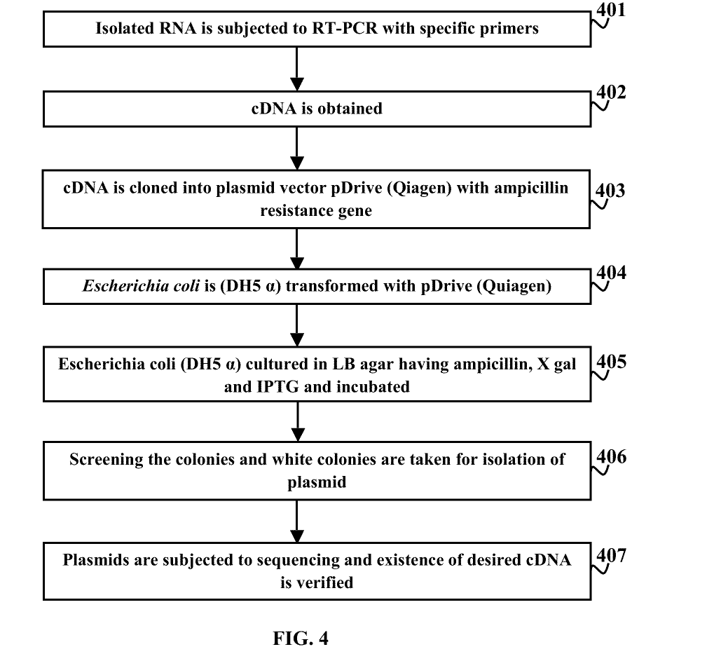

FIG. 4 illustrates a flow chart explaining a method of cloning and screening of transformed Escherichia coli, according to one embodiment herein. Before proceeding with the isolated RNA, the presence of 18srRNA is confirmed by subjecting the 18srRNA to RT-PCR with the primers (401). After RT-PCR, the complementary DNA (cDNA) is obtained (402). The cDNA is cloned with ampicillin resistance gene into plasmid vector pDrive (Qiagen) (403). After the cloning of the plasmid vector pDrive (Qiagen), it is transformed into Escherichia coli strain number DH5 (404). The transformed Escherichia coli is cultured on the LB media (Luria bertani media) or selection media having the X-gal (5-bromo-4-chloro-indolyl-β-D-galactopyanoside), the IPTG (isopropyl β-D-1-thiogalactopyranoside) and the ampicillin (405). After culturing the transformed Escherichia coli on the selection media, the transformed colonies are screened and the white colonies are taken for isolating the plasmid (406). Further the isolated plasmids are subjected to sequencing for verifying the presence of desired cDNA (407).

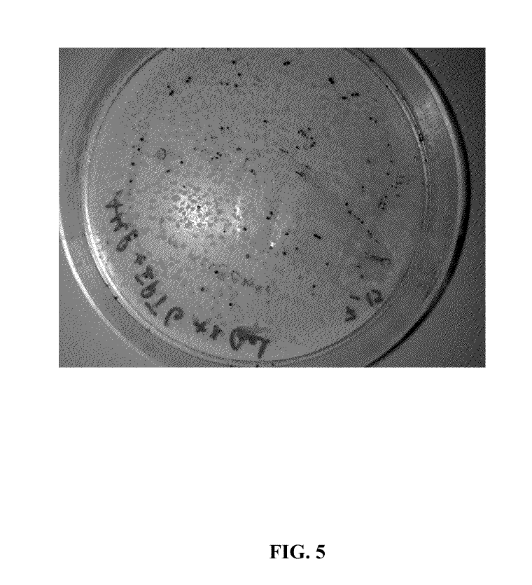

FIG. 5 shows an image exhibiting the white blue colony screening of 18srRNA gene after transformation, according to one embodiment herein. The cells transformed with the plasmid vector containing cDNA produce white colonies and the cells transformed with non-recombinant plasmid vector (only plasmid vector without cDNA) grow into blue colonies.

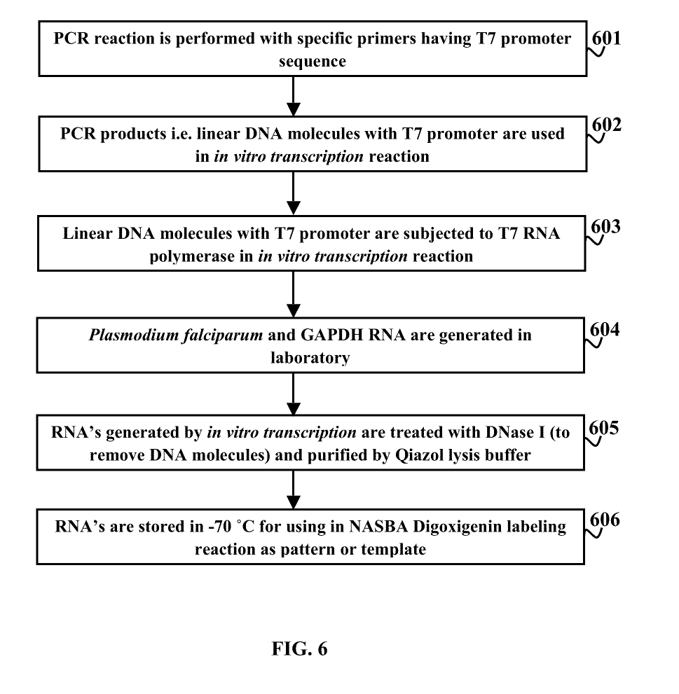

FIG. 6 illustrates a flow chart indicating in vitro transcription for generating RNA, according to one embodiment herein. After confirming the presence of the cDNA of 18srRNA in the plasmids, the cDNA is subjected to in vitro transcription for generating the RNA. The main reason for in vitro transcription is to generate RNA with same size. The cDNA is subjected to polymerase chain reaction (PCR reaction). The PCR is performed with specific primers for cDNA of each of 18srRNA and GAPDH of Plasmodium falciparum, Plasmodium vivax and healthy human blood respectively having T7 promoter sequence (601). The PCR products or linear DNA molecules obtained with T7 promoter are used in in vitro transcription reaction (602). The linear DNA molecules with T7 promoter are subjected to T7 RNA polymerase in the in vitro transcription (603). The RNA for Plasmodium falciparum, Plasmodium vivax and GAPDH are generated in laboratory by this method (604). The RNA generated for the Plasmodium falciparum, Plasmodium vivax and the GAPDH are treated with a DNase I (to remove DNA molecules) and purified by Qiazol lysis buffer (605). The RNA for Plasmodium falciparum, Plasmodium vivax and GAPDH are stored in −70° C. for using in NASBA digoxigenin labeling reaction as pattern or template strand (606).

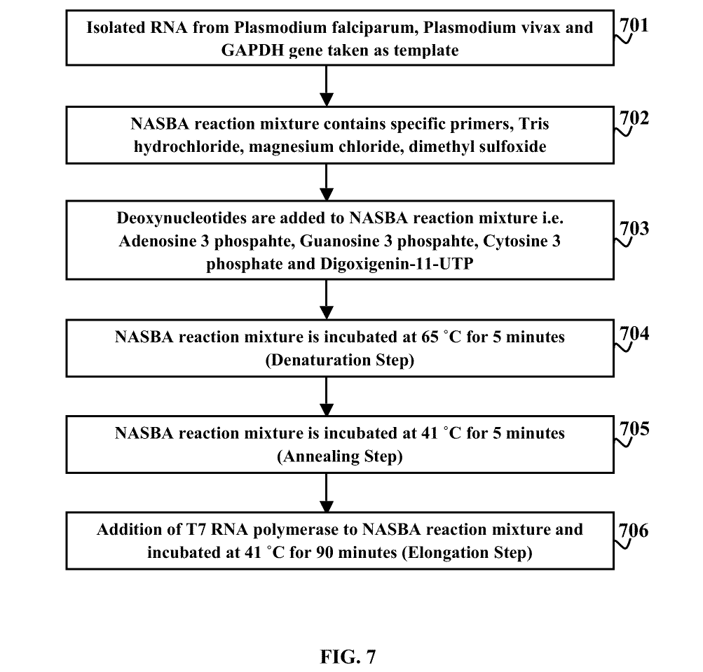

FIG. 7 illustrates a flow chart indicating the Digoxigenin-11-UTP labeling of NASBA products i.e. RNA, according to one embodiment herein. The digoxigenin labeling of the isolated RNA is performed. The isolated 18srRNA from Plasmodium falciparum (180bp), Plasmodium vivax (160bp) and GAPDH from blood of healthy human are taken as template (701). The final reaction volume for NASBA reaction mixture is 21 μl and comprise of specific primers of 100 μl for each RNA and GAPDH, 1 μg of template RNA, 40 mM Tris hydrochloride with a pH of 8.5 (Fermentas, Germany), 4 mM magnesium chloride (Fermentas, Germany), Dimethyl sulfoxide with 15% w/v (Fermentas, Germany), 10 units of M-MuL V RT (Fermentas, Germany), 22 units of T7 RNA polymerase (Fermentas, Germany) and 12 units of Ribolock (Fermentas, Germany) (702). Then 5 mM of deoxy nucleotide 3 phospahte (Fermentas, Germany), 10 mM of Adenosine 3 phospahte, Guanosine 3 phospahte, Thiamidine 3 phospahate (Fermentas, Germany), 6.5 mM UTP (Fermentas, Germany), 3.5 mM Digoxigenin-11-UTP (Roche, Germany) are added to the reaction mixture (703). The NASBA reaction mixture is then subjected to incubation for 5 minutes at 65° C. and this causes a denaturation of the RNA strand (704). The NASBA reaction mixture is then incubated for 5 minutes at 41° C. and this leads to an annealing of specific primers with the template RNA (705).The T7 RNA polymerase is now added in the NASBA reaction mixture and incubated for 90 minutes for 41° C. and this leads to an elongation of the complementary chains of RNA (706). Hence the digoxigenin labeled 18sRNA of Plasmodium vivax, Plasmodium falciparum and GAPDH RNA are amplified.

The primers used are:

Plasmodium vivax 18srRNA reverses primer
AATTCTAATACGACTCACTATAGGGGGAGTAACAAGGACTTCCAAGCC
Plasmodium vivax 18srRNA forward primer
TTAATCCACATAACTGATACTTCG
GAPDH reverse primer
5′AATTCTAATACGACTCACTATAGGGGAAGATGGTGATGGGA
TTTC-3′
GAPDH forward primer
5′-CATTGACCTCAACTACATGG-3′
Plasmodium falciparum 18srRNA forward primer
5′-GAGTACACTATATTCTTATTTGAA-3′
Plasmodium falciparum 18srRNA reverse primer
5′ATTCTAATACGACTCACTATAGGGAGAGCAAGTACATTCTTA
AAAGA-3′

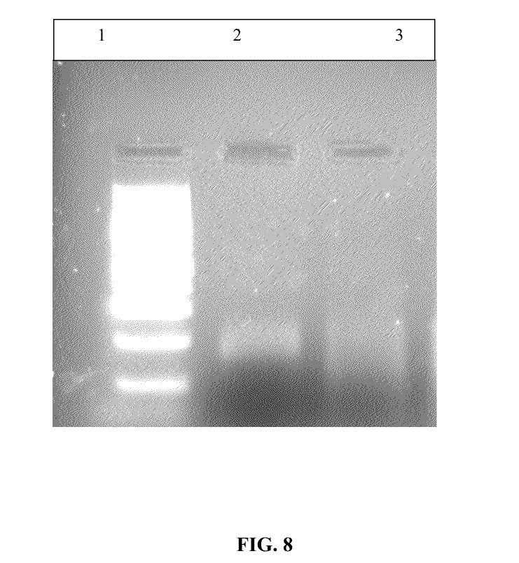

FIG. 8 shows an image indicating an agarose gel electrophoresis of the Plasmodium falciparum 18srRNA and the nucleic acid sequence based amplification (NASBA) and dig-labeling of the P. falciparum 18srRNA, according to one embodiment herein. With respect to FIG. 8, the well 1 of the agarose gel is loaded with the DNA marker. The well 2 of the agarose gel is loaded with the NASBA 18srRNA not labeled with digoxigenin and the well 3 of the agarose gel is loaded with NASBA digoxigenin labeled 18srRNA.

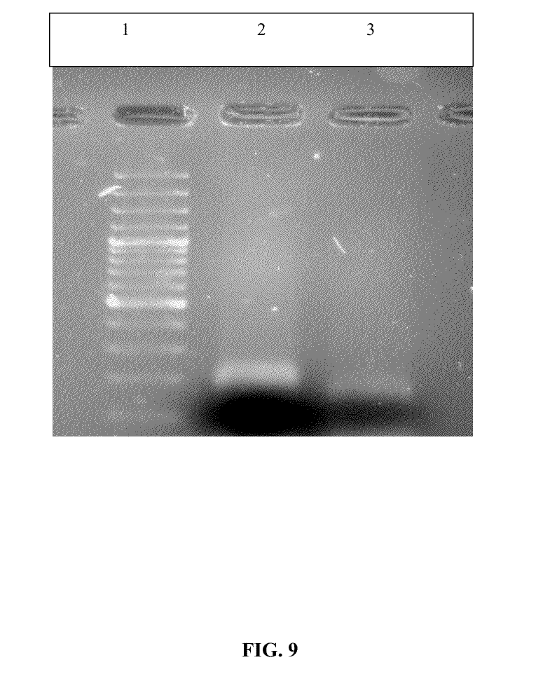

FIG. 9 shows an image indicating an agarose gel electrophoresis of NASBA for Plasmodium falciparum 18srRNA and GAPDH, according to one embodiment herein. With respect to FIG. 9, the well 1 of the agarose gel is loaded a DNA marker. The well 2 of the agarose gel is loaded with the NASBA product 18srRNA of Plasmodium falciparum and the well 3 of the agarose gel is loaded with NASBA product GAPDH of a healthy human.

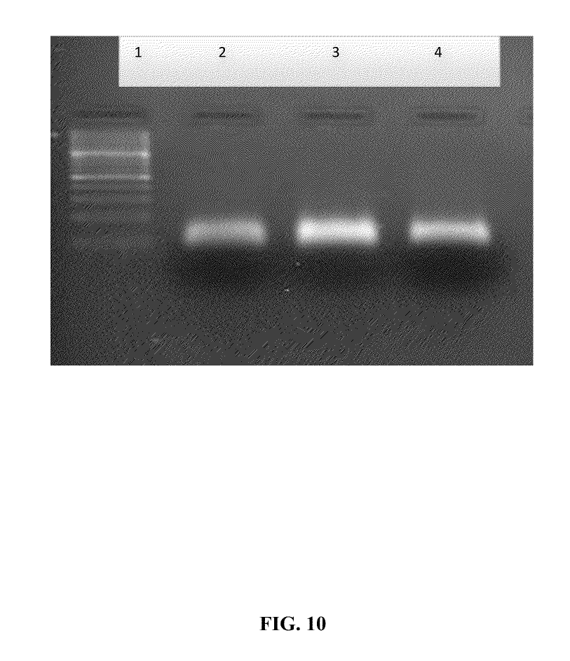

FIG. 10 shows an image exhibiting an agarose gel electrophoresis of invitro transcription of GAPDH gene, according to one embodiment herein. With respect to FIG. 10, the well 1 of the agarose gel is loaded with DNA marker. The well 2 of the agarose gel is loaded with GAPDH gene obtained after increasing an amount of NTP mix (nucleotide tri phosphate mix) in the in vitro transcription reaction. The well 3 of the agarose gel is loaded with the GAPDH gene obtained after increasing an amount of the T7 RNA polymerase in the in vitro transcription reaction. The well 4 of the agarose gel is loaded with the GAPDH gene obtained after increasing an amount of GAPDH obtained after PCR.

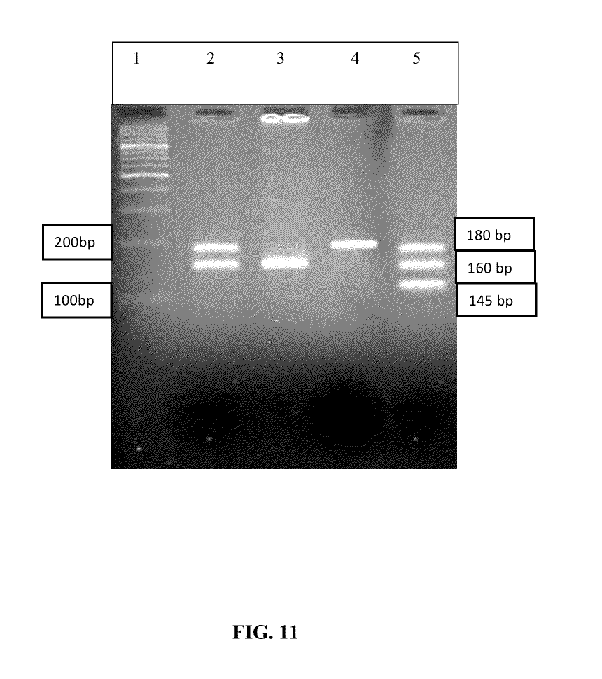

FIG. 11 shows an image exhibiting the agarose gel electrophoresis of 18srRNA of Plasmodium vivax, Plasmodium falciparum and the GAPDH isolated from healthy human blood obtained after RT-PCR, according to one embodiment herein. With respect to FIG. 11, the well 1 of the agarose gel is loaded with the DNA marker, and two bands of the DNA marker comprising 200 by and 100 by DNA are seen. The well 2 of the agarose gel is loaded with the Plasmodium falciparum 18srRNA and Plasmodium vivax 18srRNA obtained after performing RT-PCR. The 18srRNA of Plasmodium falciparum and Plasmodium vivax have a molecular size of 180 by and 160 by respectively. The well 3 of the agarose gel is loaded with the Plasmodium vivax 18srRNA obtained after performing the RT-PCR, and has a molecular size of the RNA is 160 bp. The well 4 of the agarose gel is loaded with the Plasmodium falciparum 18srRNA obtained after performing RT-PCR, and has a molecular size of 200 bp. The well 5 of the agarose gel is loaded with the Plasmodium falciparum 18 srRNA, Plasmodium vivax 18 srRNA and human GAPDH obtained after performing RT-PCR, the molecular sizes of the 18srRNA and GAPDH are 180 bp, 160 by and 145 by respectively.

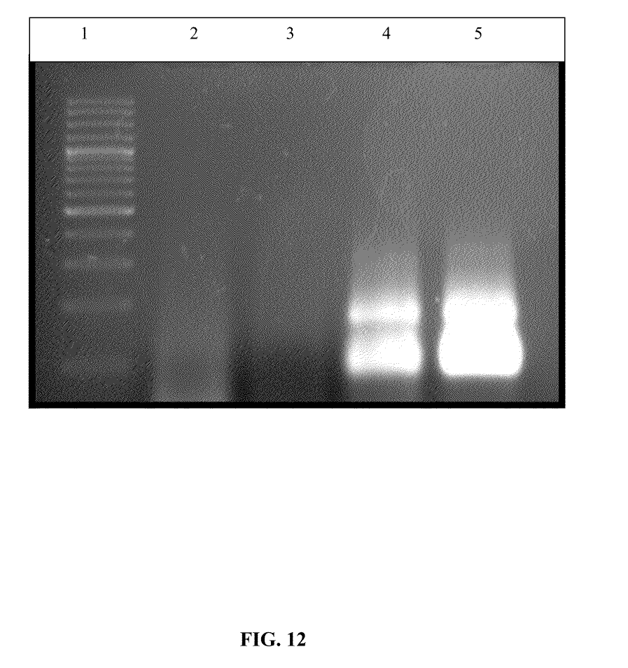

FIG. 12 shows an image exhibiting the agarose gel electrophoresis of GAPDH, the Plasmodium falciparum 18srRNA and the Plasmodium vivax 18srRNA obtained after NASBA, according to one embodiment herein. The dimethyl sulfoxide (DMSO) is used in PCR to inhibit secondary structures in the DNA template or the DNA primers. The DMSO is added to the PCR mix, where DMSO interferes with the self-complementarity of the DNA, thereby minimizing interfering reactions. With respect to FIG. 12, the well 1 of the agarose gel is loaded with the DNA marker. The well 2 of the agarose gel is loaded with 18srRNA of Plasmodium falciparum, Plasmodium vivax and GAPDH of the healthy human obtained from RT-PCR wherein the RT-PCR reaction mixture conatains 5% DMSO. The well 3 of the agarose gel is loaded with 18srRNA of the Plasmodium falciparum, Plasmodium vivax and the GAPDH of the healthy human obtained from a RT-PCR wherein the RT-PCR reaction mixture contains 10% DMSO. The well 4 of the agarose gel is loaded with 18srRNA of Plasmodium falciparum, Plasmodium vivax and GAPDH of the healthy human obtained from RT-PCR wherein the RT-PCR reaction mixture contains 20% DMSO. The well 5 of the agarose gel is loaded with 18srRNA of Plasmodium falciparum, Plasmodium vivax and GAPDH of the healthy human obtained from RT-PCR wherein the RT-PCR reaction mixture contains 15% DMSO.

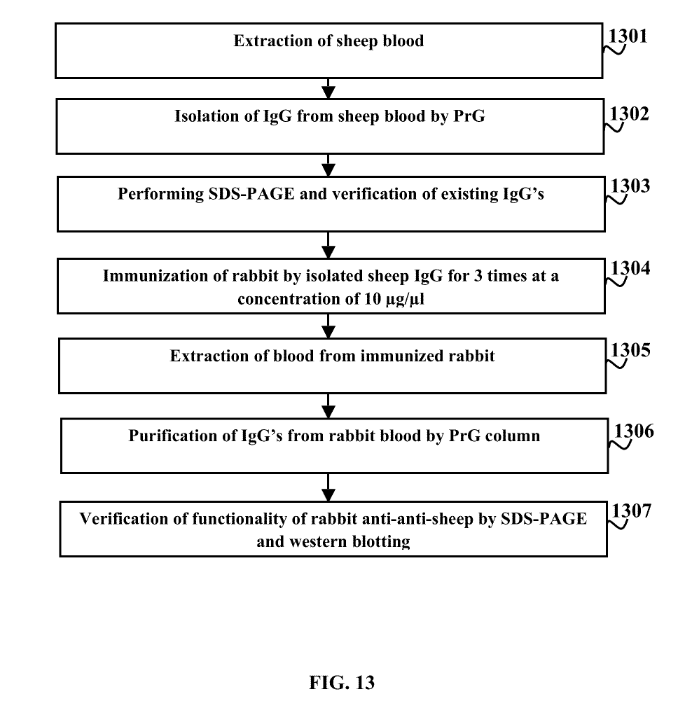

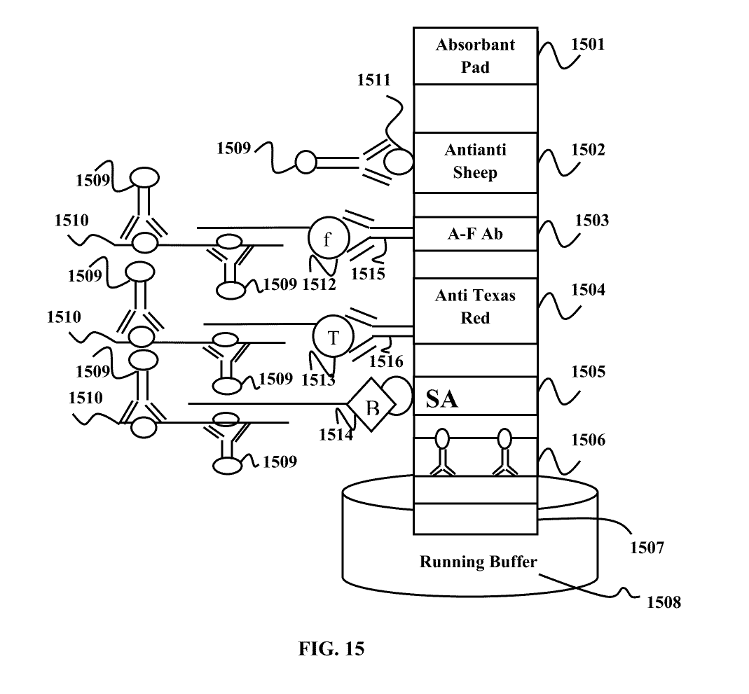

FIG. 13 illustrates a flow chart explaining an process of anti anti-sheep preparation for loading on dipstick nano-biosensor, according to one embodiment herein. Before the assembly of the dipstick nano-biosensor, the capture reagents are prepared. The capture reagents are the reagents which develop the color lines on the dipstick nano-biosensor upon interacting with the malaria parasite antigen. The anti digoxigenin antibody is generated in a sheep. The anti digoxigenin antibody is conjugated to nano gold particles (Ref FIG. 15) in laboratory by physical adsorption for 1.5 hours in cold room by continuous mixing of gold nanoparticles. The capture reagents include a streptavidine, an anti fluoresceine and an anti Texas Red, and specific probes for the 18srRNA of Plasmodium falciparum, Plasmodium vivax and the GAPDH of healthy human are brought from Roche and Biolegend Company respectively. The GAPDH probes are conjugated with fluoresceine and the 18srRNA probe of Plasmodium falciparum is conjugated to biotin.

GAPDH probe:
5′GAGAACGGGAAGCTTGTCATCTTTTTTTTTTTTTTT-
FLUORESCEIN-3′
Plasmodium falciparum 18srRNA probe:
CATAGGTAACTATACATTTATTCAAAAAAAAAAAAAAA-Biotin

The anti anti-sheep was prepared in laboratory. The anti anti-sheep was then conjugated to nano gold particles and used in dipstick nano-biosensor assembly. The blood from a sheep is extracted (1301). The IgG's (Immunoglobin G) are isolated from the sheep blood by a PrG column to obtaing an isolate (1302).The sodium dodecyl sulfate polyacrylamide gel electrophoresis (SDS-PAGE) is performed for verifying the presence of IgG in the isolate (1303). After SDS-PAGE, a 3 kg rabbit is selected and immunized with sheep IgG, for 3 times at a concentration of 10 μg/μl (1304). After the rabbit is immunized, the blood is extracted from the immunized rabbit (1305). The immunized rabbit produces antibody against the sheep IgG's and the antibodies are called as anti anti-sheep IgG. The IgG's from rabbit blood are purified by PrG column (1306). The purified IgG's from the rabbit blood are subjected to SDS-PAGE and Western blotting for verifying functionality (1307).

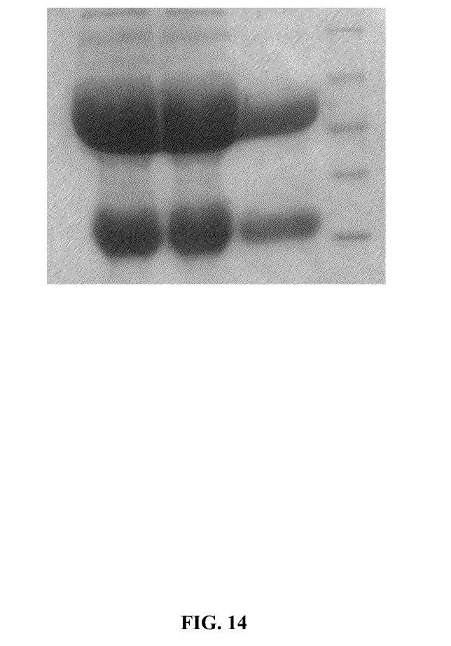

FIG. 14 shows an image exhibiting sodium dodecyl sulphate polyacrylamide gel electrophoresis (SDS-PAGE) of sheep IgG's after purification by PrG column before immunizing rabbit, according to one embodiment herein. The heavy chain and light chain of the anti anti sheep IgG are separated by using 2-ME (2-mercaptoethanol) in the SDS-PAGE procedure. The 2-mercaptoethanol is a reducing agent which denatures IgG by breaking the disulphide linkages.

FIG. 15 shows a schematic view or representation of the dipstick nano-biosensor assembly, according to one embodiment herein. The different regions of the dipstick nanobiosensor are represented in FIG. 15. The absorbent pad 1501 is made up of a cellulose membrane. The second control line 1502 comprises anti anti-sheep coated on a nitrocellulose membrane. The second control line is further attached with digoxigenin 1511. The first control line 1503 comprising antifluoresceine antibody 1515 is coated on nitrocellulose membrane. The second test line 1504 comprises antitexas red 1516 coated on nitrocellulose membrane. The first test line 1505 comprising streptavidin is coated on nitrocellulose membrane. The conjugate pad 1506 comprising the gold nanoparticle conjugated to the anti digoxigenin 1509 is coated on the fiberglass. The wicking pad 1507 is made of a cellulose membrane. The running buffer 1508 is located at the bottom of the nanobiosensor dipstick. The gold nanoparticle conjugated with anti digoxigenin 1509 is present along with the NASBA product 1510 enriched by the digoxigenin 1511. Further the digoxigenin is present along with gold nanoparticle conjugated with anti digoxigenin 1509 on the second control line 1502. The fluoresceine 1512 is present conjoined with the anti fluoresceine antibody 1515 on the first control line 1503. The Texas red 1513 is present conjoined with the anti Texas red 1515 on the second test line 1512. The biotin 1514 is conjoined with streptavidin on the first test line 1505. The antifluorescein antibody 1515 is coated on the first control line 1503. The anti Texas red 1516 is coated on the second test line 1504.

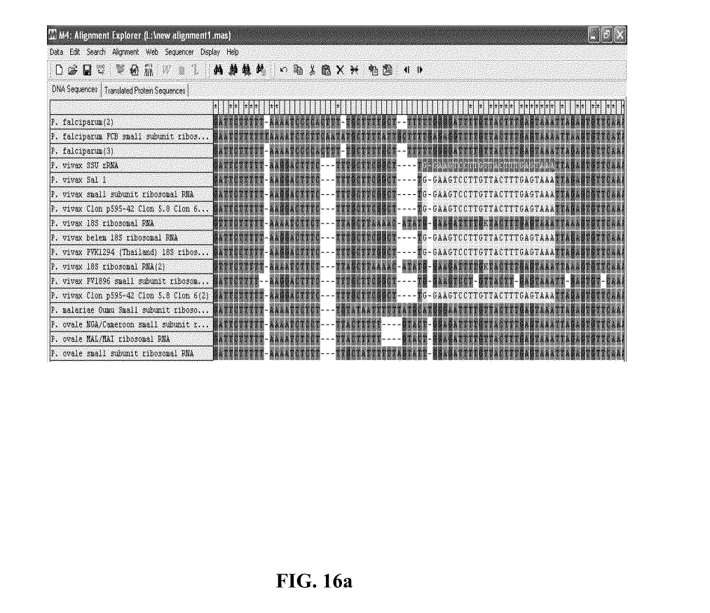

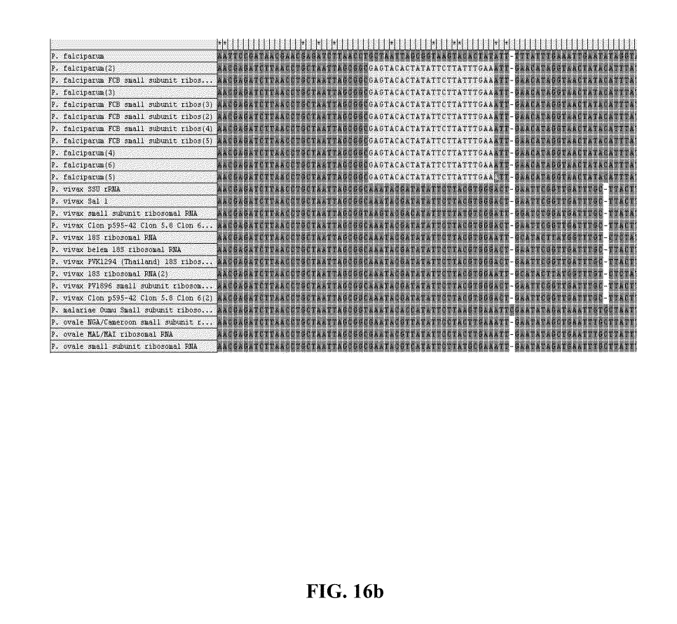

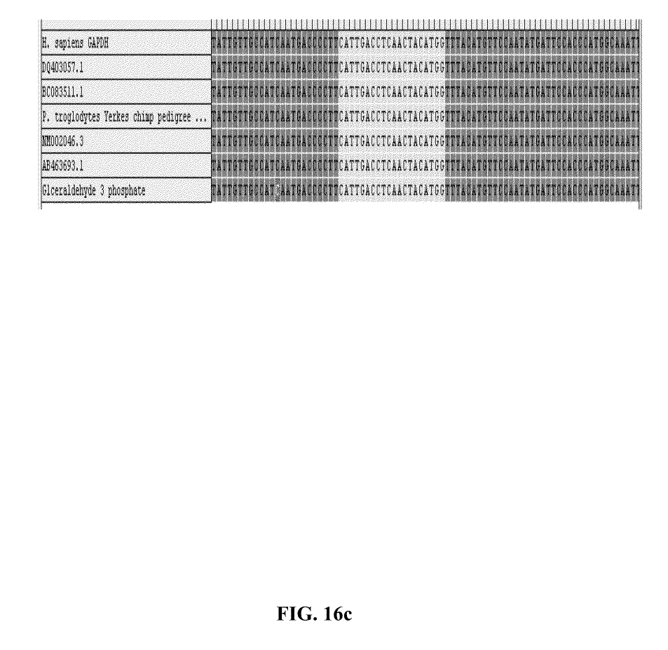

FIG. 16a illustrates a screen shot indicating a designing of the specific primers for the Plasmodium vivax, according to one embodiment herein. The primer designed detects specific sequences of the Plasmodium vivax in in vitro transcription.

FIG. 16b illustrates a screen shot indicating a designing of the specific primers for Plasmodium vivax, according to one embodiment herein. The primer designed detects specific sequences of Plasmodium falciparum in in vitro transcription.

FIG. 16c illustrates a screen shot indicating a designing of the specific primers for human GAPDH, according to one embodiment herein. The primer designed detects specific sequences of GAPDH in invitro transcription.

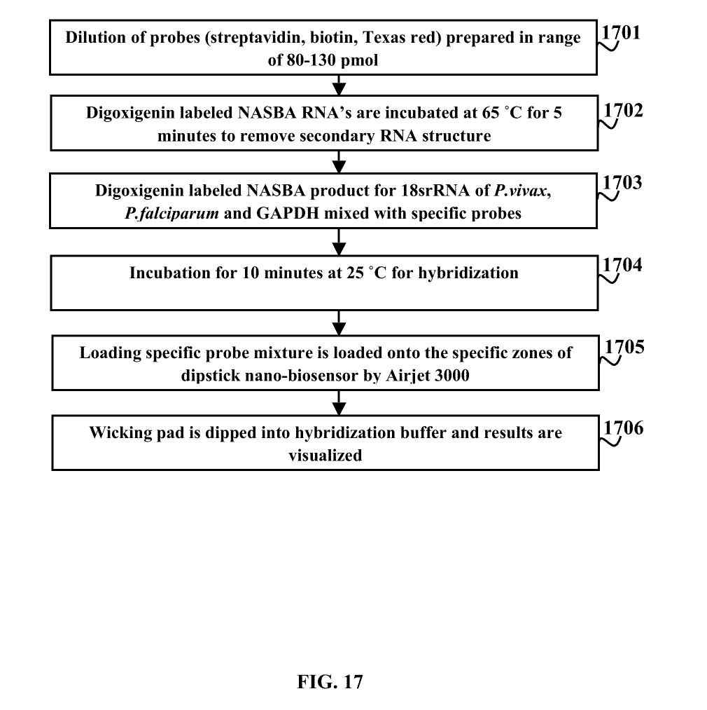

FIG. 17 illustrates a flow chart explaining the oligochromatography, according to one embodiment herein. For the diagnosis of the malarial parasites, the dipstick nano-biosensor is subjected to oligochromatography. The probes (sterptavidin, Texas red and Biotin) are prepared in the range of 80-130 pmol concentration (1701). The digoxigenin labeled RNA's are incubated at 65° C. for 5 minutes to remove the secondary RNA structure (1702). The digoxigenin labeled NASBA 18srRNA of Plasmodium falciparum, Plasmodium vivax and the GAPDH of healthy human are mixed with specific probes (1703). In the first test zone, a streptavidin,and a biotin are bound to the Plasmodium falciparum digoxigenin labeled NASBA product and the GAPDH. Further anti digoxigenin conjugated gold nano-particles and streptavidin are coated in this zone. In the second test zone, the anti texas red and the texas red are bound to the Plasmodium vivax digoxigenin labeled NASBA product. Further anti digoxigenin conjugated to gold nano-particles and anti texas red are coated in this zone. The first control zone comprises an antifluoresceine, a fluoresceine bound to GAPDH probe and digoxigenin labeled GAPDH. Further this zone comprises the anti digoxigenin conjugated to gold nano-particles. The second control zone comprises the anti anti-sheep bounded to digoxigenin conjugated to gold nano-particles. (Ref FIG. 15). After specific probes are mixed with specific probes, the probe mixture for each zone of dipstick nano-biosensor is incubated at 25° C. for 10 minutes (1704). After incubation, the probe mixture for each specific zone is loaded onto the specific zone of dipstick nano-biosensor by Airjet 3000 (1705). After all zones of the dipstick nano-biosensor are loaded with specific probe mixture, the wicking pad of dipstick nano-biosensor is dipped into hybridization buffer and the results are visualized (1706). The composition of hybridization buffer as follows:

| Ingredients | Amount needed |
|---|---|
| BSA | 0.1% |
| Na₂HPO₄ | 0.09% |
| NaH₂PO₄ | 0.045% |
| NaCl | 0.9% |
| Tween 20 | 0.001% |

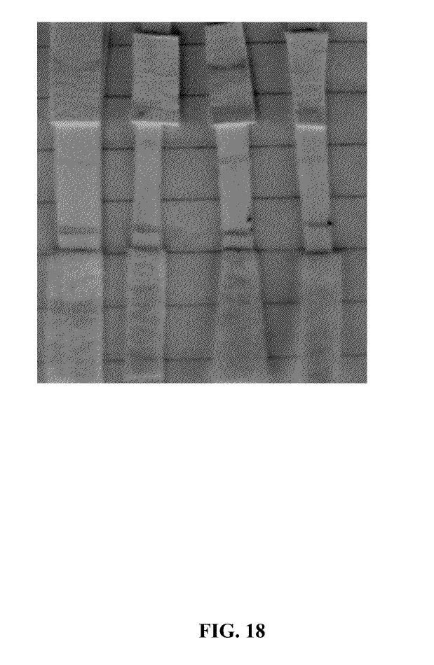

FIG. 18 shows an image exhibiting the first test line and second control line in dipstick nano-biosensor, according to one embodiment herein. With respect to FIG. 18, the red line at the bottom of the dipstick shows that the dipstick could detect hybridization product of Plasmodium falciparum 18srRNA and specific probe. The red line at the top of dipstick shows that the gold nano-particles conjugated to anti digoxigenin react with anti anti-sheep coated on the second control line.

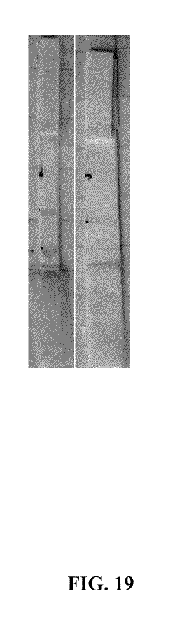

FIG. 19 shows an image exhibiting the hybridization product of Plasmodium falciparum 18srRNA and the human GAPDH, according to one embodiment herein. With respect to FIG. 19, a line is formed in first test zone and also at the first and second control zones. The diagnosis is positive for a malaria patient.

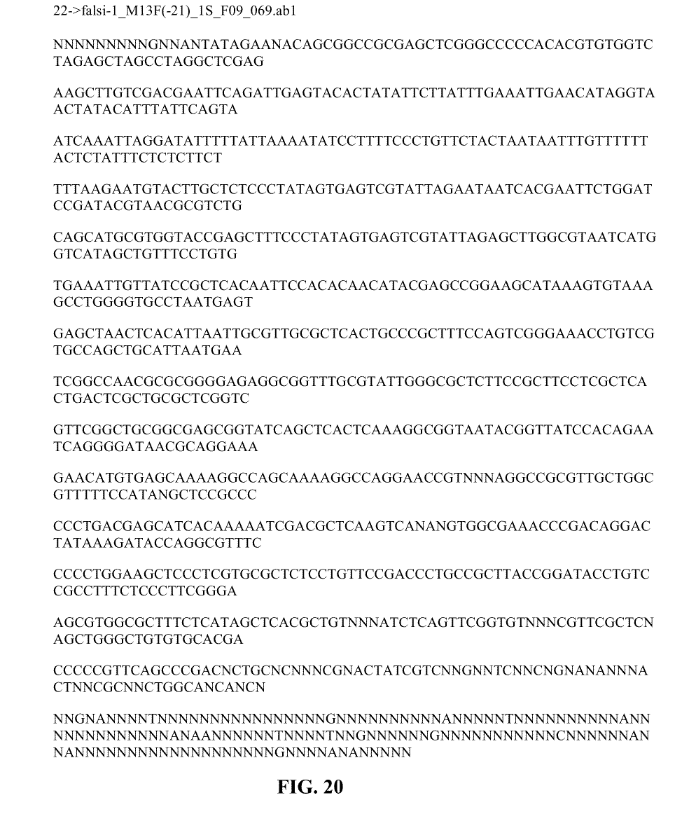

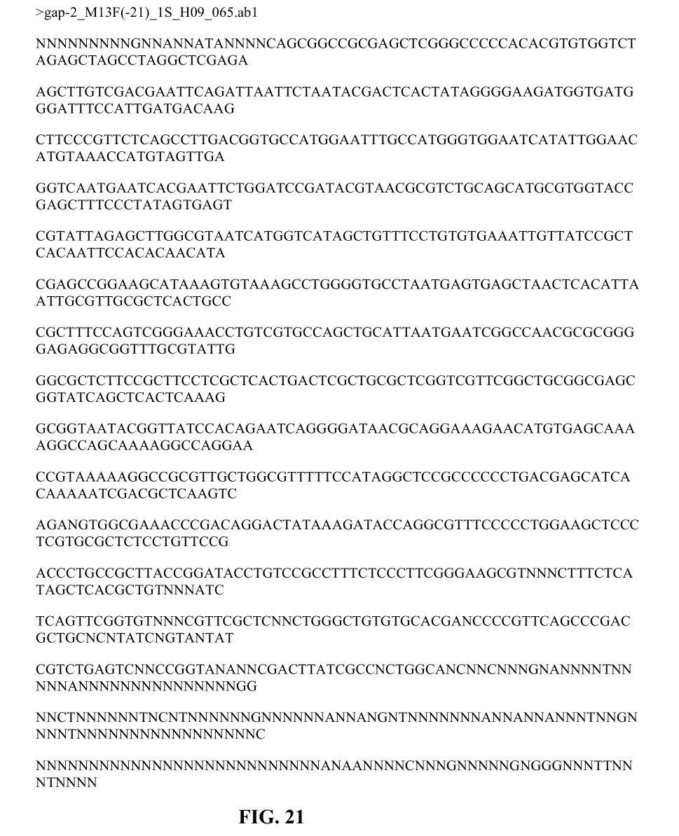

FIG. 20 shows a result of a Plasmodium falciparum 18srRNA gene sequencing results, while FIG. 21 shows a result of human GAPDH gene sequencing according to one embodiment herein.

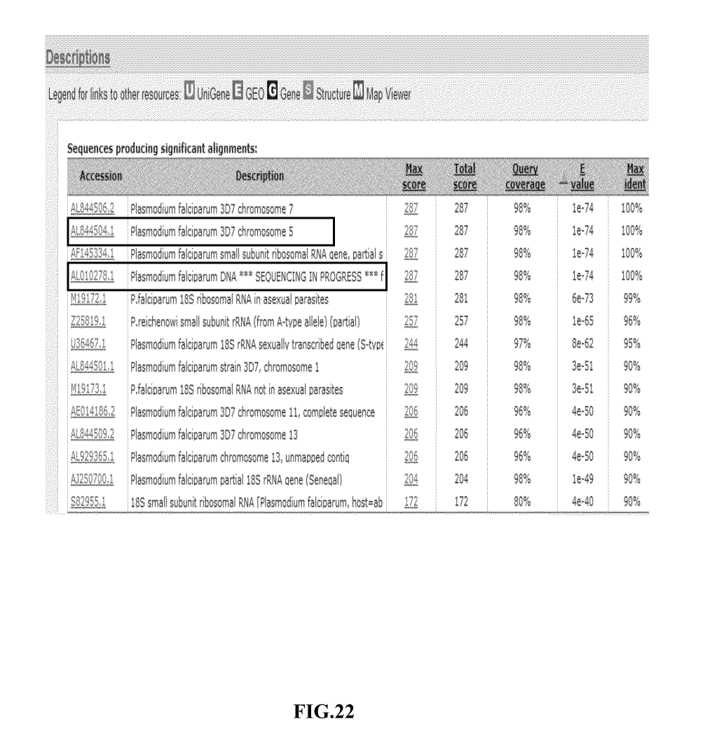

FIG. 22 shows a screen shot illustrating a Blast analysis of amplified sequence of Plasmodium falciparum 18srRNA, according to one embodiment herein. The Blast results show that the 18srRNA sequence of the Plasmodium falciparum aligned with the Plasmodium falciparum 3D7 chromosome 5 and the Plasmodium falciparum DNA *** Sequencing in progress.

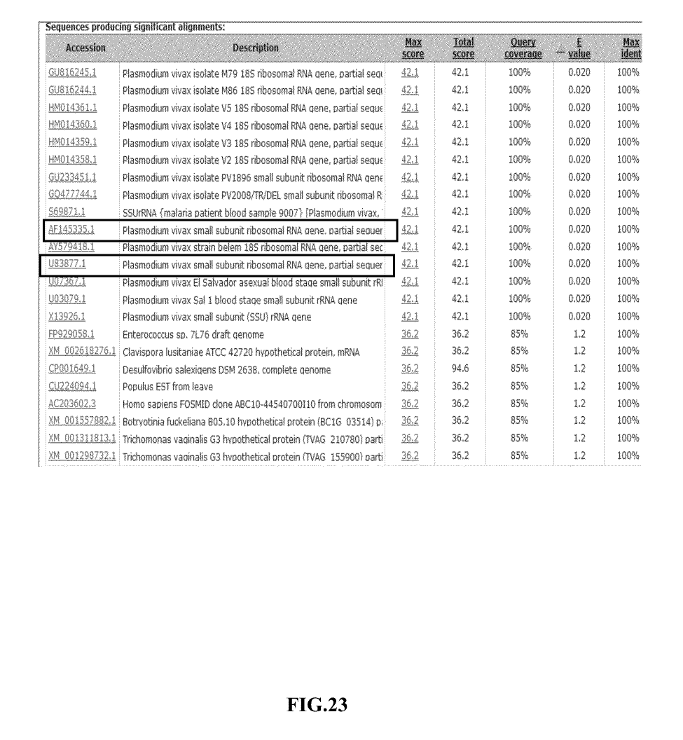

FIG. 23 shows a screen shot illustrating a Blast analysis of amplified sequence of Plasmodium vivax 18srRNA, according to one embodiment herein. The Blast results show that the 18srRNA sequence of Plasmodium vivax aligned with the Plasmodium vivax small subunit ribosomal RNA gene.partial sequence and the Plasmodium vivax small subunit ribosomal RNA gene, partial sequence.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation.

Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A dipstick nano-biosensor for detecting Plasmodium vivax and Plasmodium falciparum, the dipstick nano-bio sensor comprising:
a cellulose membrane;
a nitrocellulose membrane;
a fibreglass; and
a plurality of probes;
wherein the cellulose membrane, the nitrocellulose membrane and the fiber glass are coated on a backing plastic plate coated with gold nanoparticles conjugated with an anti-body and wherein the anti-body is an anti-digoxigenin.

2. The dipstick nano-biosensor according to claim 1, wherein the cellulose and the nitrocellulose membrane are coated on the backing plastic plate with an overlap of 1 mm.

3. The dipstick nano-biosensor according to claim 1, wherein the gold nanoparticles are conjugated to the anti digoxigenin antibody by a physical adsorption for 1.5 hours in a cold room by a continuous mixing of the gold nanoparticles, and wherein a size of the gold nanoparticles is 0.40-25 nm.

4. The dipstick nano-biosensor according to claim 1, wherein the plurality of the probes comprise a streptavidin, a texas red, a biotin and a fluoresceine.

5. The dipstick nano-biosensor according to claim 1, wherein the backing plastic plate comprises three regions and wherein the three regions comprise a wicking pad, a conjugate pad, and an absorbent pad respectively.

6. The dipstick nano-biosensor according to claim 5, wherein the conjugate pad comprises a test zone and a control zone, and wherein the test zone comprises a first test line and a second test line for detecting Plasmodium falciparum and Plasmodium vivax respectively, and wherein the control zone comprises a first control zone and a second control line for checking a correctness of NASBA and oligo chromatography respectively.

7. The dipstick nano-biosensor according to claim 6, wherein the first test line comprises a nitrocellulose membrane coated with a streptavidin conjugated to a biotin and wherein the second test line comprises a nitrocellulose membrane coated with an anti texas red.

8. The dipstick nanobiosnsor according to claim 6, wherein the first control line comprises a nitrocellulose membrane coated with an antifluorescene antibody and wherein the second control line comprises a nitrocellulose membrane coated with an anti anti-sheep.

9. The dipstick nano-biosensor according to claim 1, wherein the plurality of probes comprises a 18srRNA and wherein the 18srRNA is isolated from Plasmodium falciparum, Plasmodium vivax and a GAPDH, and wherein the GAPDH is isolated from a blood of healthy human individual, and wherein the isolated 18srRNA and the GAPDH are subjected to an agarose gel electrophoresis to extract specific bands with sequences from an agarose gel.

10. The dipstick nano-biosensor according to claim 9, wherein the 18srRNA is subjected to a Reverse Transcriptase Polymerase Chain Reaction (RT-PCR) to get a cDNA.

11. The dipstick nano-biosensor according to claim 9, wherein the isolated sequences from the agarose gel are cloned into a plasmid vector, and wherein the cloned plasmid vectors are induced into an Escherichia coli, and wherein the plasmids are sequenced.

12. The dipstick nano-biosensor according to claim 9, wherein 18srRNA and GAPDH are subjected to Nucleic Acid Sequence Based Amplification (NASBA), and wherein NASBA amplified 18sRNA and GAPDH are labelled with digoxigenin-11-UTP.

13. The dipstick nano-biosensor according to claim 1, wherein the backing plastic plate is coated with digoxigenin-11-UTP labelled 18srRNA, streptavidin, anti texas red, biotin and fluorescein by Airjet 3000.

14. A method of synthesizing dipstick nano-biosensor for detecting Plasmodium vivaxand Plasmodium falciparum, the method comprises:
culturing Plasmodium falciarum and Plasmodium vivax;
extracting RNA from Plasmodium falciparum and Plasmodium vivax culture and a healthy donor;
cloning and sequencing extracted RNA using a Reverse Transcriptase Polymerase Chain Reaction (RT-PCR) to get a cDNA;
performing In vitro transcription of extracted RNA;
preparing control positive RNA;
isolating GAPDH from a blood of healthy human individual;
setting and performing NASBA-DIG labelling of both Pasmodium falciparum and Plasmodium vivax and GAPDH;
selecting a plurality of membranes for assembling a dipstick, and wherein the plurality of membranes includes a cellulose membrane, a nitrocellulose membrane, and a fibreglass; and
conjugating nano gold particles to anti digoxigenin antibody to form a dipstick.


SEQUENCE LISTING (8 sequences)

SEQ ID NO: 1 - DNA, 48 bp, Artificial Sequence
  Synthetic Plasmodium vivax 18srRNA reverses primer
aattctaatacgactcactatagggggagtaacaaggacttccaagcc

SEQ ID NO: 2 - DNA, 24 bp, Artificial Sequence
  Synthetic Plasmodium vivax 18srRNA forward primer
ttaatccacataactgatacttcg

SEQ ID NO: 3 - DNA, 45 bp, Artificial Sequence
  Synthetic GAPDH reverse primer
aattctaatacgactcactataggggaagatggtgatgggatttc

SEQ ID NO: 4 - DNA, 20 bp, Artificial Sequence
  Synthetic GAPDH forward primer
cattgacctcaactacatgg

SEQ ID NO: 5 - DNA, 24 bp, Artificial Sequence
  Synthetic Plasmodium falciparum 18srRNA forward primer
gagtacactatattcttatttgaa

SEQ ID NO: 6 - DNA, 47 bp, Artificial Sequence
  Synthetic Plasmodium falciparum 18srRNA reverse primer
attctaatacgactcactatagggagagcaagtacattcttaaaaga

SEQ ID NO: 7 - DNA, 36 bp, Artificial Sequence
  Synthetic GAPDH probe
gagaacgggaagcttgtcatcttttttttttttttt

SEQ ID NO: 8 - DNA, 38 bp, Artificial Sequence
  Synthetic Plasmodium falciparum 18srRNA probe
cataggtaactatacatttattcaaaaaaaaaaaaaaa